(19) United States
(12) Patent Application Publication
(10) Pub. No.: US 2005/0287183 A1
(43) Pub. Date: Dec. 29, 2005

(54) Cosmetic composition comprising at least one apolar wax and a dispersion of polymer particles in a fatty phase

(76) Inventor: Caroline Lebre, Thiais (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/147,155

(22) Filed: Jun. 8, 2005

Related U.S. Application Data
(60) Provisional application No. 60/580,104, filed on Jun. 17, 2004.

(30) Foreign Application Priority Data
Jun. 8, 2004 (FR) 04 06170

(51) Int. Cl. A61K 7/00

(52) U.S. Cl. 424/401

(57) ABSTRACT

Disclosed herein is a cosmetic composition comprising polymer particles dispersed in a fatty phase, and at least 5% by weight of at least one apolar wax with a melting point of less than 65° C., relative to the total weight of the composition. Also disclosed herein is a cosmetic composition comprising a fatty phase containing at least 5% by weight of at least one non-volatile hydrocarbon-based oil relative to the total weight of the composition, and polymer particles dispersed in the fatty phase, the composition comprising at least one apolar wax with a melting point of less than 65° C. Further disclosed herein is a cosmetic composition comprising i) polymer particles dispersed in a fatty phase, and ii) at least one wax, wherein the dynamic viscosity of the composition measured at 25° C., using a Mettler RM 180 rotary viscometer, ranges from 0.1 to 120 Pa.s, and the amount of the at least one wax is greater than or equal to 15% by weight relative to the total weight of the composition.

This application claims benefit of U.S. Provisional Application No. 60/580,104, filed Jun. 17, 2004, the contents of which are incorporated herein by reference. This application also claims benefit of priority under 35 U.S.C. § 119 to French Patent Application No. 04 06170, filed Jun. 8, 2004, the contents of which are also incorporated by reference.

Disclosed herein is a composition which may be used in the cosmetics field, comprising polymer particles dispersed in a fatty phase. This composition may also comprise an apolar wax whose melting point is less than or equal to 65° C.

More specifically, the present disclosure relates to a care and/or makeup composition for keratin materials, for instance the skin of the human face and/or of the human body, including the scalp; the integuments, for instance the eyelashes, the eyebrows, the nails, and the hair; and also human lips, the lower eyelids, and the upper eyelids.

The composition disclosed herein may make it possible to obtain makeup with good staying power, which is not tacky, does not migrate and feels comfortable.

As used herein, the term “makeup with good staying power” means a makeup whose color and/or gloss persists throughout the day, and which withstands ordeals, such as meals in the case of a lipstick.

The present inventor has discovered, surprisingly, that by combining a minimum amount of at least one low-melting apolar wax with a fatty phase containing a dispersion of polymer particles, a makeup with good staying power that is not tacky, does not migrate, and feels comfortable may be obtained.

The present inventor has also discovered, surprisingly, that it may be possible to obtain such a makeup by combining the at least one apolar wax in the presence of a minimum amount of non-volatile hydrocarbon-based oil and/or by limiting the amount of volatile oil in the composition. This is all the more surprising since, in the prior art, the staying power of cosmetic compositions may be improved by incorporating into the composition a large proportion of at least one volatile oil.

Lip makeup formulations, such as lipsticks and lip glosses, may be appreciated for their glossy result.

These compositions may be based on gelled or non-gelled oils and on pasty compounds. The oils chosen may have a high refractive index and high viscosity, so as to ensure the desired gloss effect. However, these oils may also make the film tacky, which may make the lipstick uncomfortable over time. Since the predominant constituents of these formulations are liquid, they may have a tendency to migrate beyond the lips by escaping their surrounding fine lines. The staying power of these formulations may thus be disappointing.

The present inventor has discovered, surprisingly, that by combining a low-melting apolar wax with a fatty phase containing a dispersion of polymer particles, a glossy makeup that also has good staying power, is not tacky, does not migrate, and feels comfortable may be obtained.

The present inventor has also discovered, surprisingly, that by combining at least one low-melting apolar wax with a fatty phase containing a dispersion of polymer particles, a cosmetic composition containing a high proportion of at least one wax that is in the form of a paste, rather than of a stick, may be obtained.

In certain embodiments, the composition disclosed herein may make it possible to obtain non-tacky continuous deposits that give good coverage, do not migrate, and do not dry out the skin and/or the lips onto which it is applied, either during the application or over time. It may also have good application properties, since the deposit may dry quickly and the color may deposit correctly in a single application.

The staying power and migration-resistance properties may make the composition disclosed herein, for example, suitable for making skin makeup products such as foundations. By modifying the gloss properties, the compositions disclosed herein may be suitable, for example, for making makeup products for the lips, such as lipsticks and/or lip glosses; for the eyes, such as mascara, eyeliners, and eyeshadows; and for the skin.

The present disclosure relates, as noted, to makeup products for the lips, and also to lipcare and/or lip treatment products, for instance balms; makeup products for the skin, including the scalp, for instance daily care creams and antisun creams for facial skin; makeup products for the skin, for example the human face and/or the human body, for instance foundations cast, for example in a stick or a dish, concealer products, and temporary tattoo products; body hygiene products, for instance deodorants, such as deodorants in stick form, and eye makeup products, for instance eyeliners, such as eyeliners in pencil form, and mascaras, such as mascaras in cake form.

One embodiment disclosed herein is a cosmetic composition comprising polymer particles dispersed in a fatty phase, and at least 5% by weight of at least one apolar wax with a melting point of less than 65° C., relative to the total weight of the said composition.

Further disclosed herein is a cosmetic composition comprising a fatty phase comprising at least 5% by weight of non-volatile hydrocarbon-based oil relative to the total weight of the composition, and polymer particles dispersed in the fatty phase, the composition containing at least one apolar wax with a melting point of less than 65° C.

Another embodiment disclosed herein relates to a composition comprising a fatty phase free of volatile oil or comprising less than 50% by weight of at least one volatile oil, relative to the weight of the fatty phase, the fatty phase comprising at least one apolar wax with a melting point of less than 65° C.

Yet another embodiment disclosed herein relates to a cosmetic composition comprising i) polymer particles dispersed in a fatty phase, and ii) at least one wax, wherein:
the dynamic viscosity of the composition measured at 25° C., using a Mettler RM 180 rotary viscometer, ranges from 0.1 to 120 Pa.s, and wherein
the amount of the at least one wax is greater than or equal to 15% by weight relative to the total weight of the composition.

As used herein, the term “fatty phase” means any non-aqueous medium.

In certain embodiments, the fatty phase is at least partially liquid at room temperature (25° C.) and atmospheric pressure (760 mmHg) and comprises at least one fatty substance that is liquid at room temperature, also known as at least one oil.

The dynamic viscosity of the composition may be measured using a Mettler RM 180 viscometer. The Mettler RM 180 machine (Rheomat) may be equipped with various rotors depending on the order of magnitude of the viscosity that it is desired to measure. For a viscosity ranging from 0.18 to 4.02 Pa.s, the machine may be equipped with a No 3 rotor. For a viscosity ranging from 1 to 24 Pa.s, the machine may be equipped with a No 4 rotor, and for a viscosity ranging from 8 to 122 Pa.s, the machine may be equipped with a No 5 rotor. The viscosity is read on the machine in deviation units (DU). Reference is then made to tables supplied with the measuring machine in order to obtain the corresponding value in poises.

The spin speed of the rotor is 200 rpm.

From the moment at which the rotor is set in rotation, at a constant imposed spin speed (in the present case 200 rpm), the viscosity value of the composition may vary over time. Measurements are taken at regular time intervals until they become constant. The viscosity value that becomes constant over time is the value taken as being the dynamic viscosity of the composition disclosed herein. According to the measuring system 75 set up on the machine, the viscosity measurement is taken after 10 minutes.

The viscosity of the composition may range from 0.5 to 50 Pa.s, such as from 1 to 30 or from 2 to 20 Pa.s.

The composition may be obtained according to a blending process. Two types of machine may be used to perform the blending: a roll mill comprising two rolls rotating in opposite directions, between which the paste passes, and a mixer-screw extruder. A mixer-extruder may be used, so that a paste of constant quality is obtained in a reproducible manner. Furthermore, by adapting the outlet die of the mixer-extruder, it may be possible to package the composition in-line at the outlet of the mixer-extruder.

According to certain embodiments, a mixer-extruder-cooker may be used, comprising, in an outer envelope equipped at the outlet with an extrusion die, one or two shafts driven in rotation such that the peripheral structure of one shaft co-operates with the outer envelope and, where appropriate, with the peripheral structure of the other shaft, to ensure the mixing and blending of the paste and its displacement in the envelope towards the extrusion die.

According to another embodiment, the process disclosed herein is such that a mixture of the non-pulverulent constituents of the composition is introduced into the top of a mixer-screw extruder, at a temperature at which the at least one wax is molten, and the pulverulent constituents of the composition are introduced into the said mixer-screw extruder at at least one point before the extrusion die. In this regard, the content of European Patent Application No. EP 667 146 is incorporated herein by reference.

In certain embodiments, the polymer is such that, when it is dispersed in sufficient amount in the fatty phase, the mean gloss at 20° of a deposit of the composition, once spread onto a support, is greater than or equal to 30 out of 100.

As used herein, the term “mean gloss” means the gloss as may be conventionally measured using a glossmeter by the following method.

A coat ranging from 50 μm to 150 μm in thickness of the composition is spread using an automatic spreader onto a Leneta brand contrast card of reference Form 1A Penopac. The coat covers at least the white background of the card. When the composition is solid, it is melted, if necessary, on the card after it has been spread out so that it recovers the white background.

The deposit is left to dry for 24 hours at a temperature of 30° C., and the gloss at 20° is then measured on the white background using a Byk Gardner brand glossmeter of reference microTri-Gloss.

This measurement (ranging from 0 to 100) is repeated at least three times, and the mean gloss is the mean of the at least three measurements taken.

The mean gloss of the composition measured at 20° may be greater than or equal to 30, greater than or equal to 35, greater than or equal to 40, greater than or equal to 45, greater than or equal to 50 out of 100, greater than or equal to 55, or greater than or equal to 60.

In certain embodiments, the mean gloss of the composition, once spread onto a support, measured at 60° is greater than or equal to 50, greater than or equal to 60, greater than or equal to 65, greater than or equal to 70, greater than or equal to 75, greater than or equal to 80, greater than or equal to 85, or greater than or equal to 90 out of 100.

The mean gloss at 60° is measured as described above, by taking the measurement at 60° rather than at 20°.

According to one embodiment, the mean gloss of the composition measured at 20° may be greater than or equal to 35, such as greater than or equal to 40, 45, or 50 out of 100, and/or the mean gloss of the composition measured at 60° may be greater than or equal to 65, such as greater than or equal to 70 or 75 out of 100. In this embodiment, the composition may constitute a liquid lipstick.

Also disclosed herein is the cosmetic use of the composition defined above for improving the gloss of a deposit on the skin and/or the lips and/or the integuments, such as a makeup.

According to one embodiment of the composition disclosed herein, the polymer is such that, when it is in sufficient amount in the composition, the composition is capable of forming a deposit that has a wear index of greater than or equal to 30%, such as greater than or equal to 40%, greater than or equal to 45%, or greater than or equal to 50%.

The wear index of the deposit obtained with the composition disclosed herein may be determined according to the measuring protocol described below.

A support (40 mm×70 mm rectangle) comprising an acrylic coating (hypoallergenic acrylic adhesive on polyethylene film sold under the name Blenderme ref. FH5000-55113 by the company 3M Santé) bonded onto a layer of polyethylene foam that is adhesive on the face opposite the one to which the adhesive plaster is fixed (foam layer sold under the name RE 40X70EP3 from the company Joint Technique Lyonnais Ind.) is prepared.

The color L*₀a*₀b*₀ of the support, on the acrylic coating face, is measured using a Minolta CR300 calorimeter.

The support thus prepared is preheated on a hotplate maintained at a temperature of 40° C. so that the surface of the support is maintained at a temperature of 33° C.±1° C.

While leaving the support on the hotplate, the composition is applied to the entire non-adhesive surface of the support (i.e., to the surface of the acrylic coating), spreading it out with a brush to obtain a deposit of the composition of about 15 μm, and this deposit is then left to dry for 10 minutes.

After drying, the color L*a*b* of the film thus obtained is measured.

The color difference ΔE1 between the color of the film and the color of the naked support is then determined via the following relationship:
ΔE1=\sqrt{(L*−L₀*)²+(a*−a₀*)²+(b*−b₀*)²}

The support is then bonded via its adhesive face (adhesive face of the foam layer) to an anvil 20 mm in diameter and equipped with a screw pitch. A sample of the support/deposit assembly is then cut out using a sample punch 18 mm in diameter. The anvil is then screwed onto a press (Statif Manuel Imada® SV-2 from the company Someco) equipped with a tensile testing machine (Imada® DPS-20 from the company Someco).

A strip 33 mm wide and 29.7 cm long is drawn on a sheet of white photocopier paper with a basis weight of 80 g/m², a first line is marked 2 cm from the edge of the sheet, and a second line is then marked 5 cm from the edge of the sheet, the first and second lines thus delimiting a box on the strip; next, a first mark and a second mark located in the strip at reference points 8 cm and 16 cm, respectively, from the second mark, are applied. 20 μl of water are placed on the first mark, and 10 μl of refined sunflower oil (sold by the company Lesieur) are placed on the second mark.

The white paper is placed on the base of the press, and the sample placed on the box of the strip of paper is then pressed at a pressure of about 300 g/cm² exerted for 30 seconds. The press is then opened and the sample is again placed just after the second mark (i.e., next to the box), a pressure of about 300 g/cm² is again exerted, and the paper is displaced, in a rectilinear manner as soon as the contact is made, at a speed of 1 cm/s over the entire length of the strip such that the sample passes through the water and oil deposits.

After removing the sample, some of the deposit has transferred onto the paper. The color L*′, a*′, b*′ of the deposit remaining on the sample is then measured.

The color difference ΔE2 between the color of the deposit remaining on the sample and the color of the naked support is then determined via the following relationship
ΔE2=\sqrt{(L*′L₀*)²+(a*′a₀*)²+(b*′b₀*)²}

The wear index of the composition, expressed as a percentage, is equal to the ratio:
100×ΔE2/ΔE1

The measurement is performed on 6 supports in succession, and the wear index corresponds to the mean of the six measurements obtained with the six supports.

Disclosed herein is the cosmetic use of the composition defined above for giving gloss and migration resistance to a deposit on the skin and/or the lips and/or the integuments, such as a makeup deposit.

Polymer in Dispersion

According to certain embodiments, the polymer is a solid that is insoluble in the fatty phase of the composition at room temperature, for example at around 25° C. The polymer is also insoluble in the fatty phase at its softening point, unlike a wax, which is soluble in the fatty phase at a temperature above its melting point. In this sense, the polymer is not a wax.

In at least one embodiment, the polymer in dispersion also allows the formation of a deposit that is continuous and homogeneous and/or is characterized by the overlapping of the polymer chains.

The composition disclosed herein may, for example, comprise at least one stable dispersion of essentially spherical polymer particles of at least one polymer, in the fatty phase.

These dispersions may, for example, be in the form of polymer nanoparticles in stable dispersion in the liquid organic phase. The nanoparticles may have a mean size ranging from 5 to 800 nm, such as ranging from 50 to 500 nm. However, it is possible to obtain polymer particles ranging up to 1 μm in size.

In certain embodiments, the polymer particles in dispersion are insoluble in water-soluble alcohols, for instance ethanol.

The polymers in dispersion that may be used in the composition disclosed herein may, in at least one embodiment, have a molecular weight ranging from 2,000 to 10,000,000 g/mol and a Tg ranging from −100° C. to 300° C., such as from −50° C. to 100° C. or from −10° C. to 50° C.

The polymer may, for example, be film-forming. It is possible to use film-forming polymers having a low Tg, for example a Tg less than or equal to skin temperature, such as a Tg less than or equal to 40° C.

As used herein, the term “film-forming polymer” means a polymer that is capable, by itself or in the presence of an auxiliary film-forming agent, of forming a continuous film that adheres to a support, such as to keratin materials, and may be a cohesive film, such as a film whose cohesion and mechanical properties are such that the film can be isolated from the support.

However, it is also possible to use a non-film-forming polymer. As used herein, the term “non-film-forming polymer” means a polymer that is incapable by itself of forming an isolable film.

Among the film-forming polymers that may be mentioned are acrylic or vinyl free-radical homopolymers or copolymers, for example polymers with a Tg of less than or equal to 40° C., such as ranging from −10° C. to 30° C., used alone or as a mixture.

Among the non-film-forming polymers that may be mentioned are optionally crosslinked vinyl or acrylic free-radical homopolymers or copolymers, for example with a Tg of greater than 40° C., such as ranging from 45° C. to 150° C., used alone or as a mixture.

As used herein, the term “free-radical polymer” means a polymer obtained by polymerization of unsaturated, such as ethylenic, monomers, each monomer being capable of homopolymerizing (unlike polycondensates). The free-radical polymers may be vinyl polymers or copolymers, and for example may be acrylic polymers.

The acrylic polymers may result from the polymerization of ethylenically unsaturated monomers containing at least one acid group and/or esters of these acid monomers and/or amides of these acids.

Meth(acrylic)/methacrylate copolymers, such as acrylic/acrylate copolymers such that the mass ratio of the acrylic units and of the acrylate units ranges from 0.1% to 40%, such as from 2% to 30% or from 5% to 20%, may be used in the context of certain embodiments.

Monomers bearing an acid group that may be used include α,β-ethylenic unsaturated carboxylic acids such as acrylic acid, methacrylic acid, crotonic acid, maleic acid, and itaconic acid. (Meth)acrylic acid and crotonic acid may be used, for example, and in at least one embodiment, (meth)acrylic acid is used.

The acid monomer esters may be chosen from (meth)acrylic acid esters (also known as (meth)acrylates), for instance alkyl (meth)acrylates, such as of a C₁-C₂₀ or C₁-C₈ alkyl, aryl (meth)acrylates, such as of a C₆-C₁₀ aryl, and hydroxyalkyl (meth)acrylates, such as of a C₂-C₆ hydroxyalkyl. Alkyl (meth)acrylates that may be mentioned include methyl, ethyl, butyl, isobutyl, 2-ethylhexyl, and lauryl (meth)acrylate. Hydroxyalkyl (meth)acrylates that may be mentioned include hydroxyethyl (meth)acrylate and 2-hydroxypropyl (meth)acrylate. Aryl (meth)acrylates that may be mentioned include benzyl and phenyl acrylate.

The (meth)acrylic acid esters that may be mentioned include the alkyl (meth)acrylates.

Free-radical polymers that may be used include copolymers of (meth)acrylic acid and of alkyl (meth)acrylate, such as of a C₁-C₄ alkyl. Methyl acrylates optionally copolymerized with acrylic acid may be used.

Amides of the acid monomers that may be mentioned include (meth)acrylamides, such as N-alkyl(meth)acrylamides, for example of a C₂-C₁₂ alkyl, such as N-ethylacrylamide, N-t-butylacrylamide, and N-octylacrylamide; and N-di(C₁-C₄)alkyl(meth)-acrylamides.

The acrylic polymers may also result from the polymerization of ethylenically unsaturated monomers containing at least one amine group, in free form or in partially or totally neutralized form, or alternatively in partially or totally quaternized form. Such monomers may be chosen from, for example, dimethylaminoethyl (meth)acrylate, dimethylaminoethylmethacrylamide, vinylamine, vinylpyridine, and diallyldimethylammonium chloride.

The vinyl polymers may also result from the homopolymerization or copolymerization of at least one monomer chosen from vinyl esters and styrene monomers. In particular, these monomers may be polymerized with acid monomers and/or esters thereof and/or amides thereof, such as those mentioned above. Examples of vinyl esters that may be mentioned include vinyl acetate, vinyl propionate, vinyl neodecanoate, vinyl pivalate, vinyl benzoate, and vinyl t-butylbenzoate. Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

As other vinyl monomers that may be used, mention may also be made of:
N-vinylpyrrolidone, N-vinylcaprolactam, vinyl-N-(C₁-C₈)alkylpyrroles, vinyloxazoles, vinylthiazoles, vinylpyrimidines, and vinylimidazoles, and
olefins such as ethylene, propylene, butylene, isoprene, and butadiene.

The vinyl polymer may be crosslinked with at least one difunctional monomer, for example a difunctional monomer comprising at least two ethylenic unsaturations, such as ethylene glycol dimethacrylate and/or diallyl phthalate.

In a non-limiting manner, the polymers in dispersion disclosed herein may be chosen from at least one of the following polymers or copolymers: polyurethanes; polyurethane-acrylics; polyureas; polyurea-polyurethanes; polyester-polyurethanes; polyether-polyurethanes; polyesters; polyesteramides; alkyds; acrylic polymers; acrylic copolymers; vinyl polymers; vinyl copolymers; acrylic-silicone copolymers; polyacrylamides; silicone polymers, for instance silicone polyurethanes and silicone acrylics; and fluoro polymers.

The at least one polymer in dispersion in the liquid fatty phase may represent, as solids, from 5% to 40%, such as from 5% to 35% or from 8% to 30% by weight, relative to the total weight of the composition.

According to one embodiment, the polymer particles in dispersion are surface-stabilized with a stabilizer that is solid at room temperature. In this case, the amount of solids in the dispersion represents the total amount of polymer plus stabilizer, given that the amount of polymer cannot be less than 5%.

In at least one embodiment, a dispersion of particles of at least one film-forming polymer is used.

Stabilizer

The polymer particles in organic medium are surface-stabilized, as the polymerization proceeds, by means of a stabilizer chosen from block polymers, grafted polymers, random polymers, and mixtures thereof. The stabilization may take place by any known means, such as by direct addition of the block polymer, grafted polymer, and/or random polymer during the polymerization.

The stabilizer may also be present in the mixture before polymerization. However, it is also possible to add it continuously, for example when the monomers are also added continuously.

2-30% by weight, such as 5-20% by weight of stabilizer relative to the initial monomer mixture may be used.

When a grafted polymer and/or a block polymer is used as stabilizer, the synthesis solvent may be chosen such that at least some of the grafts or blocks of the polymer-stabilizer are soluble in the solvent, the rest of the grafts or blocks being insoluble therein. The polymer-stabilizer used during the polymerization should be soluble, or dispersible, in the synthesis solvent. Furthermore, a stabilizer whose insoluble blocks or grafts have a certain affinity for the polymer formed during the polymerization may be chosen.

Among the grafted polymers that may be mentioned are silicone polymers grafted with a hydrocarbon-based chain and hydrocarbon-based polymers grafted with a silicone chain.

Grafted copolymers having, for example, an insoluble polyacrylic skeleton with soluble poly(12-hydroxystearic acid) grafts of are also suitable for use.

Thus, grafted or block copolymers comprising at least one polyorganosiloxane block and at least one block of a free-radical polymer, for instance grafted acrylic/silicone copolymers, may thus be used, which may be used, for instance, when the synthesis medium and then the organic phase of the first composition contains a silicone phase.

It is also possible to use grafted-block or block copolymers comprising at least one polyorganosiloxane block and at least one block of a polyether. The polyorganopolysiloxane block may be chosen from polydimethylsiloxane and poly(C₂-C₁₈)alkylmethylsiloxane. The polyether block may be a poly(C₂-C₁₈)alkylene, such as polyoxyethylene and polyoxypropylene. For example, dimethicone copolyols and (C₂-C₁₈)alkyl dimethicone copolyols such as those sold under the name Dow Corning 3225C by the company Dow Corning, and lauryl methicones such as those sold under the name Dow Corning Q2-5200 by the company Dow Corning, may be used.

Grafted-block or block copolymers that may also be mentioned include those comprising at least one block resulting from the polymerization of at least one ethylenic monomer containing at least one optionally conjugated ethylenic bonds, for instance ethylene and dienes such as butadiene and isoprene, and of at least one block of a vinyl polymer or a styrene polymer. When the ethylenic monomer comprises several optionally conjugated ethylenic bonds, the residual ethylenic unsaturations after the polymerization may be hydrogenated. Thus, in a known manner, the polymerization of isoprene leads, after hydrogenation, to the formation of an ethylene-propylene block, and the polymerization of butadiene leads, after hydrogenation, to the formation of an ethylene-butylene block. Among these polymers that may be mentioned are block copolymers, such as diblock copolymers and triblock copolymers, for example polystyrene/polyisoprene (SI), polystyrene/polybutadiene (SB) such as those sold under the name Luvitol® HSB by BASF, of the type such as polystyrene/copoly(ethylene-propylene) (SEP) such as those sold under the name Kraton® by Shell Chemical Co. and of the type such as polystyrene/copoly(ethylene-butylene) (SEB). Kraton® G1650 (SEBS), Kraton® G1651 (SEBS), Kraton® G1652 (SEBS), Kraton® G1657X (SEBS), Kraton® G1701X (SEP), Kraton® G1702X (SEP), Kraton® G1726X (SEB), Kraton® D-1101 (SBS), Kraton® D-1102 (SBS), and Kraton® D-1107 (SIS) may be mentioned. The polymers may be known as hydrogenated or non-hydrogenated diene copolymers.

Gelled Permethyl® 99A-750, 99A-753-59 and 99A-753-58 (mixture of triblock and of star polymer); Versagel® 5960 from Penreco (triblock plus star polymer); and OS129880, OS129881, and OS84383 from Lubrizol (styrene/methacrylate copolymer) may also be used.

As grafted-block or block copolymers comprising at least one block resulting from the polymerization of at least one ethylenic monomer containing at least one ethylenic bond and of at least one block of an acrylic polymer, mention may be made of poly(methyl methacrylate)/polyisobutylene diblock or triblock copolymers and grafted copolymers containing a poly(methyl methacrylate) skeleton and polyisobutylene grafts.

As grafted-block or block copolymers comprising at least one block resulting from the polymerization of at least one ethylenic monomer containing at least one ethylenic bond and of at least one block of a polymer such as a C₂-C₁₈ polyalkylene (especially polyoxyethylene and/or polyoxypropylene), mention may be made of polyoxyethylene/polybutadiene and polyoxyethylene/polyisobutylene diblock or triblock copolymers.

When a random polymer is used as stabilizer, it is chosen such that it has a sufficient amount of groups making it soluble in the intended organic synthesis medium.

Copolymers based on alkyl acrylates or methacrylates derived from C₁-C₄ alcohols and on alkyl acrylates or methacrylates derived from C₈-C₃₀ alcohols may thus be used. Mention may be made of stearyl methacrylate/methyl methacrylate copolymer.

When the synthesis medium is apolar, one may choose as stabilizer a polymer that provides the fullest possible coverage of the particles, several polymer-stabilizer chains then being adsorbed onto a particle of polymer obtained by polymerization.

In this case, for example, one may use as stabilizer either a grafted polymer or a block polymer, so as to have better interfacial activity. For example, blocks or grafts that are insoluble in the synthesis solvent may provide bulkier coverage at the surface of the particles.

When the liquid synthesis solvent comprises at least one silicone oil, the stabilizer may be chosen from grafted-block and block copolymers comprising at least one polyorganosiloxane block and at least one block chosen from free-radical polymers, polyethers, and polyesters, for instance polyoxypropylene and oxyethylene blocks.

When the liquid organic phase does not comprise any silicone oil, the stabilizer may be chosen from:
(a) grafted-block and block copolymers comprising at least one polyorganosiloxane block and at least one block chosen from free-radical polymers, polyethers, and polyesters,
(b) copolymers of alkyl acrylates or methacrylates derived from C₁-C₄ alcohols, and copolymers of alkyl acrylates or methacrylates derived from C₈-C₃₀ alcohols,
(c) grafted-block or block copolymers comprising at least one block resulting from the polymerization of at least one ethylenic monomer containing conjugated ethylenic bonds, and at least one block chosen from vinyl polymers, acrylic polymers, polyethers, and polyesters.

Diblock polymers may, for example, be used as stabilizer.

Plasticizer

When the polymer has a glass transition temperature that is too high for the intended application, a plasticizer may be combined therewith. The plasticizer may be chosen from the plasticizers usually used in the art, such as from compounds liable to be solvents for the polymer. Coalescers may thus be used in order to aid the polymer to form a continuous and homogeneous deposit.

The coalescers or plasticizers that may be used in accordance with certain embodiments include those mentioned in French Patent No. FR A 2 782 917.

The composition disclosed herein may comprise at least one ester of at least one carboxylic acid containing from 1 to 7 carbon atoms and of a polyol comprising at least four hydroxyl groups, the said ester having a molar mass of less than 5,000 g/mol.

The polyol may be chosen from monosaccharides and polysaccharides comprising 1 to 10 saccharides, such as comprising 1 to 4 or one or two saccharides.

The polyol disclosed herein may be a disaccharide. Among the disaccharides that may be mentioned are sucrose (α-D-glucopyranosyl-(1-2)-β-D-fructofuranose), lactose (β-D-galactopyranosyl-(1-4)-β-D-glucopyranose), and maltose (α-D-glucopyranosyl-(1-4)-β-D-glucopyranose).

According to one embodiment, the ester is sucrose diacetate hexakis(2-methylpropanoate).

The ester may be liquid at room temperature and atmospheric pressure. It may be present in an amount ranging from 0.1% to 25% by weight, such as from 0.5% to 15% by weight or from 3% to 15% by weight, relative to the total weight of the composition.

The mass ratio between the polymer particles and the ester of acid and polyol may range from 0.5 to 100, such as from 1 to 50, from 1 to 10, or from 1 to 5.

Fatty Phase

The fatty phase of the composition may comprise at least one cosmetically and/or dermatologically acceptable and generally physiologically acceptable oil, chosen, for example, from at least one of carbon-based oils, hydrocarbon-based oils, fluoro oils, and/silicone oils of mineral, plant or synthetic origin.

As used herein, the term “oil” means any non-aqueous medium that is liquid at room temperature (25° C.) and atmospheric pressure (760 mmHg).

The total fatty phase of the composition may be present in an amount ranging from 5% to 90%, such as from 20% to 85%, by weight relative to the total weight of the composition. It may, for example, be present in an amount of at least 30% by weight relative to the total weight of the composition.

According to one embodiment, the fatty phase is free of or contains less than 50% by weight of at least one volatile oil relative to the total weight of the fatty phase. For example, the fatty phase may contain less than 40%, such as less than 30%, less than 20%, or less than 10%, by weight of at least one volatile oil relative to the total weight of the said fatty phase.

Volatile Oil of the Fatty Phase

At least one volatile oil may be included into the fatty phase of the composition disclosed herein, provided that it represents less than 50% by weight of the fatty phase. This at least one oil may be chosen from hydrocarbon-based oils and silicone oils optionally comprising alkyl or alkoxy groups that are pendent or at the end of a silicone chain.

As used herein, the term “volatile oil” means any oil having a vapor pressure, at room temperature and atmospheric pressure, of greater than 0.02 mmHg.

As volatile silicone oils that may be used according to certain embodiments, mention may be made of linear or cyclic silicones having a viscosity at room temperature of less than 8 cSt, for example containing from 2 to 7 silicon atoms, these silicones optionally comprising alkyl or alkoxy groups of 1 to 10 carbon atoms. As volatile silicone oils that may be used, mention may be made, for example, of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, and decamethyltetrasiloxane, and mixtures thereof.

As volatile oils that may be used, C₈-C₁₆ isoalkane oils (also known as iso-paraffins), for instance isododecane, isodecane, and isohexadecane; and, for example, the oils sold under the trade names Isopar® and Permethyl®, for example isododecane (Permethyl® 99A) may be used.

In certain embodiments, the fatty phase of the composition contains less than 10% of the at least one volatile oil. For example, the fatty phase may comprise less than 5%, less than 3%, or even less than 1% of the at least one volatile oil.

Apolar or Sparingly Polar Oil

According to one embodiment, the fatty phase comprises at least one apolar or sparingly polar oil, which represents at least 5% by weight relative to the total weight of the composition. For example, the at least one apolar or sparingly polar oil of the fatty phase is a non-volatile apolar or sparingly polar hydrocarbon-based oil, which may be hydrocarbon-based.

In certain embodiments, the apolar oils have a solubility parameter δₐ=0.

As used herein, the term “polar oil” means an oil comprising chemical compounds comprising at least one polar group. The “polar groups” may be well known to those skilled in the art; they may be, for example, chosen from ionic polar groups and nonionic groups chosen from —COOH; —OH; ethylene oxide; propylene oxide; —PO₄; —NHR; and —NR₁R₂ with R₁ and R₂ optionally forming a ring and representing a linear or branched C₁ to C₂₀ alkyl or alkoxy radical.

The at least one sparingly polar oil comprises at least one oil that has a mean solubility parameter at 25° C. of: 0<δₐ<5.0 (J/cm³)^{1/2}.

The at least one highly polar oil has a mean solubility parameter δₐ according to the Hansen solubility space, at 25° C., of: δₐ≧5.0 (J/cm³)^{1/2}.

The definition and calculation of the solubility parameters in the three-dimensional Hansen solubility space are described in the article by C. M. Hansen: “The three dimensional solubility parameters” J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δₚ²+δₕ²)^{1/2}.

The parameters δₚ, δₕ, δ_{D}, and δₐ may be expressed in (J/cm³)^{1/2}.

When the oily phase is a mixture of different oils, the solubility parameters of the mixture may be determined from those of the compounds taken separately, according to the following relationships:
δ_{Dmixt}=Σxiδ_{Di};δₚₘᵢₓₜ=Σxiδₚᵢ and δₕₘᵢₓₜ=Σxiδₕᵢ
δₐₘᵢₓₜ=(δ²ₚₘᵢₓₜ+δ²ₕₘᵢₓₜ)^{1/2}
in which xi represents the volume fraction of compound i in the mixture.

A person skilled in the art may determine the amounts of each oil to obtain an oily phase that satisfies the desired criteria.

The at least one apolar or sparingly polar oil may be hydrocarbon-based. As used herein, the term “hydrocarbon-based oil” means an oil formed essentially from, or comprising, carbon and hydrogen atoms, and optionally oxygen and nitrogen atoms, and containing no silicon or fluorine atoms. It may contain at least one of alcohol, ester, ether, carboxylic acid, amine and amide groups.

The at least one apolar or sparingly polar oil may be non-volatile. As used herein, the term “non-volatile oil” means any oil having a non-zero vapor pressure at room temperature and atmospheric pressure, of less than 0.02 mmHg, such as less than 10⁻³ mmHg.

The at least one apolar or sparingly polar oil may be present in an amount of at least 5% by weight, relative to the total weight of the composition. For example, the at least one apolar or sparingly polar oil may be present in an amount ranging from 5% to 80% by weight, such as from 10% to 60% by weight or from 10% to 40% by weight, relative to the total weight of the composition.

The at least one apolar or sparingly polar oil hydrocarbon-based may be present in an amount ranging from 10% to 40% by weight, such as from 15% to 30% by weight, relative to the total weight of the composition.

In certain embodiments, the at least one apolar or sparingly polar oil is a non-volatile apolar hydrocarbon-based oil. According to one embodiment, the apolar hydrocarbon-based oil is free of hetero atoms. As used herein, the term “hetero atom” means an atom other than carbon or hydrogen.

According to one embodiment, the non-volatile apolar hydrocarbon-based oil may be chosen from linear or branched saturated alkanes.

The at least one non-volatile apolar or sparingly polar hydrocarbon-based oil may be chosen from hydrocarbon-based oils with a molar mass ranging from 300 to 900 g/mol, such as from 350 to 800 g/mol.

According to one embodiment, the non-volatile apolar hydrocarbon-based oil is chosen from linear or branched hydrocarbons such as liquid paraffin, liquid petroleum jelly, liquid naphthalene, hydrogenated polyisobutene, isoeicosane, squalane, and decene/butene copolymers, and mixtures thereof.

According to one embodiment, the fatty phase comprises from 30% to 70% by weight of at least one apolar non-volatile hydrocarbon-based oil relative to the weight of the fatty phase, such as from 40% to 60% by weight relative to the weight of the fatty phase.

As other examples of non-volatile apolar or sparingly polar oils, mention may be made of:
hydrocarbon-based oils of animal origin, for instance squalene;
hydrocarbon-based plant oils such as liquid triglycerides of fatty acids having at least 10 carbon atoms;
synthetic esters and ethers, for example of fatty acids, for instance the oils of formula R₁(CO)ₓOR₂ in which R₁ represents an acid residue containing from 2 to 29 carbon atoms with x being 0 or 1 and R₂ represents a hydrocarbon-based chain containing from 3 to 30 carbon atoms, for instance tributyl acetyl citrate, oleyl erucate, 2-octyldodecyl behenate, triisoarachidyl citrate, isocetyl stearoylstearate, octyldodecanyl stearoylstearate, n-propyl acetate, tridecyl trimellitate, diisocetyl dodecanedioleate, diisocetyl stearate, arachidyl propionate, dibutyl phthalate, propylene carbonate, octyldodecyl pentanoate; polyol esters, for instance vitamin F, sorbitan isostearate, glyceryl triisostearate, and diglyceryl triisostearate, and mixtures thereof.

The fatty phase of the composition may also comprise at least one non-volatile silicone oil chosen from:
polydimethylsiloxanes (PDMS) optionally comprising an entity chosen from C₃-C₄₀ alkyl groups, C₃-C₄₀ alkoxy chains, and phenyl radicals; the polydimethylsiloxanes comprising phenyl radicals may be chosen from phenyl trimethicones;
optionally fluorinated polyalkylmethylsiloxanes, for instance polymethyltrifluoropropyldimethylsiloxanes,
polyalkylmethylsiloxanes substituted with functional groups such as hydroxyl, thiol, and amine groups;
polysiloxanes modified with fatty acids, fatty alcohols, or polyoxyalkylenes.

In certain embodiments, the fatty phase of the composition contains less than 10% of at least one silicone oil. For example, the fatty phase contains less than 5%, less than 3%, or even less than 1 % of at least one silicone oil.

Non-Volatile Highly Polar Oil

The fatty phase may contain, besides the at least one apolar or sparingly polar oil as described above, a highly polar non-volatile oil chosen from fatty acid esters of 7 to 29 carbon atoms, for instance, diisostearyl malate; isopropyl palmitate; diisopropyl adipate; caprylic/capric acid triglycerides, for instance those sold by the company Stearineries Dubois and those sold under the names Miglyol® 810, 812 and 818 by the company Dynamit Nobel; Shea butter oil; isopropyl myristate; butyl stearate; hexyl laurate; diisopropyl adipate; isononyl isononate; 2-hexyldecyl laurate; 2-octyldecyl palmitate; 2-octyldodecyl myristate; lactate; 2-diethylhexyl succinate; 2-ethylhexyl palmitate; 2-octyldodecyl stearate; castor oil; lanolic acid esters; lauric acid esters; stearic acid esters; higher fatty alcohols (of 7 to 29 carbon atoms) such as stearyl alcohol, linoleyl alcohol, linolenyl alcohol, isostearyl alcohol, 2-octyldodecanol, decanol, dodecanol, octadecanol, and oleyl alcohol; and higher fatty acids (of 7 to 29 carbon atoms) such as myristic acid, palmitic acid, stearic acid, behenic acid, oleic acid, linoleic acid, linolenic acid, and isostearic acid, and mixtures thereof.

These non-volatile highly polar oils may be present in an amount ranging from 0.1% to 10%, such as from 1% to 5%, by weight relative to the total weight of the composition.

Oil of High Molar Mass

According to one embodiment, the liquid fatty phase may contain, besides the at least one apolar or sparingly polar oil, at least one oil of high molar mass, for example having a molar mass ranging from 650 to 10 000 g/mol.

The composition disclosed herein may comprise from 2% to 30%, such as from 5% to 25% or from 5% to 15% of at least one oil with a molar mass ranging from 650 to 10,000 g/mol, such as ranging from 750 to 7,500 g/mol.

The oil with a molar mass ranging from 650 to 10,000 g/mol may be chosen from:
polybutylenes such as Indopol® H-100 (molar mass or MM=965 g/mol), Indopol® H-300 (MM=1340 g/mol), and Indopol® H-1500 (MM=2160 g/mol), sold or manufactured by the company Amoco,
hydrogenated polyisobutylenes such as Panalane® H-300 E sold or manufactured by the company Amoco (M=1340 g/mol), Viseal 20000 sold or manufactured by the company Synteal (MM=6000 g/mol) and Rewopal® PIB 1000 sold or manufactured by the company Witco (MM=1000 g/mol),
polydecenes and hydrogenated polydecenes such as: Puresyn® 10 (MM=723 g/mol) and Puresyn® 150 (MM=9200 g/mol), sold or manufactured by the company Mobil Chemicals,
vinylpyrrolidone copolymers such as: the vinylpyrrolidone/1-hexadecene copolymer Antaron® V-216 sold or manufactured by the company ISP (MM=7300 g/mol),
esters such as:
   a) linear fatty acid esters with a total carbon number ranging from 35 to 70, for instance pentaerythrityl tetrapelargonate (MM=697.05 g/mol);
   b) hydroxylated esters such as polyglyceryl-2 triisostearate (MM=965.58 g/mol),
   c) aromatic esters such as tridecyl trimellitate (MM=757.19 g/mol),
   d) esters of branched C₂₄-C₂₈ fatty alcohol or fatty acids, such as those described in European Patent Application No. EP A 0 955 039, for example triisoarachidyl citrate (MM=1033.76 g/mol), pentaerythrityl tetraisononanoate (MM=697.05 g/mol), glyceryl triisostearate (MM=891.51 g/mol), glyceryl tris(2-decyl)tetradecanoate (MM=1143.98 g/mol), pentaerythrityl tetraisostearate (MM=1202.02 g/mol), polyglyceryl-2 tetraisostearate (MM=1232.04 g/mol), and pentaerythrityl tetrakis(2-decyl)tetradecanoate (MM=1538.66 g/mol); and
   e) diol dimer esters and polyesters, such as esters of a diol dimer and of a fatty acid, and esters of a diol dimer and of a diacid.

The esters of a diol dimer and of a monocarboxylic acid may be obtained from a monocarboxylic acid containing from 4 to 34 carbon atoms, such as from 10 to 32 carbon atoms, these acids being linear or branched, and saturated or unsaturated.

As illustrative examples of monocarboxylic acids that are suitable, mention may be made of fatty acids.

The esters of a diol dimer and of a dicarboxylic acid may be obtained from a diacid dimer derived for example from the dimerization of an unsaturated fatty acid, such as of C₈ to C₃₄, of C₁₂ to C₂₂, of C₁₈ to C₂₀, or of C₁₈.

According to one embodiment, mention may be made of the diacid dimer from which the diol dimer to be esterified is also derived.

The diol dimer esters may be obtained from a diol dimer produced by catalytic hydrogenation of a diacid dimer as described above, for example hydrogenated dilinoleic diacid.

As illustrations of diol dimer esters, mention may be made of the esters of dilinoleic diacids and of dilinoleyl diol dimers sold by the company Nippon Fine Chemical under the trade name Lusplan DD-DA5 and DD-DA7.
silicone oils such as phenylsilicones, for instance Belsil® PDM 1000 from the company Wacker (MM=9000 g/mol); and
oils of plant origin such as sesame oil (820.6 g/mol).
   Synthesis Medium for the Polymer Particles

According to one embodiment, the liquid fatty phase of the composition contains at least one oil, which is the at least one organic solvent serving as the polymerization medium for the polymer particles as described above.

The polymer dispersion may be manufactured as described, for example, in European Patent No. EP A 749 747.

According to this embodiment, a mixture comprising the initial monomers and also a free-radical initiator is prepared. This mixture is dissolved in a solvent, which is referred to in the description hereinbelow as the “synthesis solvent”.

A synthesis solvent is chosen such that the initial monomers and the free-radical initiator are soluble therein, and the polymer particles obtained are insoluble therein, such that they precipitate therefrom during their formation. For example, the synthesis solvent may be chosen from alkanes such as heptane, isododecane, and cyclohexane. The polymerization of the polymer particles may be performed in a synthesis solvent as described above, and the non-volatile hydrocarbon-based oil described above may then be added and the synthesis solvent may be selectively distilled off, provided that the non-volatile hydrocarbon-based oil is miscible with the synthesis solvent.

The monomers may be present in the synthesis solvent, before polymerization, in an amount ranging from 5% to 20% by weight relative to the total weight of the reaction mixture. The total amount of the monomers may be present in the solvent before the start of the reaction, or some of the monomers may be added gradually as the polymerization reaction proceeds.

The free-radical initiator may be chosen from azobisisobutyronitrile and tert-butylperoxy-2-ethyl hexanoate.

Wax, Pasty Compound

The composition disclosed herein may comprise at least one wax.

As the term is used herein, a wax is a lipophilic fatty compound that is solid at room temperature (25° C.), which undergoes a reversible solid/liquid change of state, having a melting point of greater than or equal to 30° C., and having an anisotropic crystal organization in the solid state. The size of the crystals is such that the crystals diffract and/or scatter light, giving the composition a cloudy, more or less opaque appearance. By bringing the wax to its melting point, it may be possible to make it miscible with oils and to form a microscopically homogeneous mixture, but, on returning the temperature of the mixture to room temperature, recrystallization of the wax in the oils of the mixture may be obtained.

According to certain embodiments, the melting point of the wax corresponds to the temperature of the most endothermic peak observed by thermal analysis (DSC) as described in standard ISO 11357-3; 1999.

The melting point of the wax may be measured using a differential scanning calorimeter (DSC), for example the calorimeter sold under the name MDSC 2920 by the company TA Instruments.

The measuring protocol is, for example, as follows:

A sample of 5 mg of wax placed in a crucible is subjected to a first temperature increase ranging from −20° C. to 100° C., at a heating rate of 10° C./minute, and is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the value of the temperature corresponding to the top of the peak of the curve representing the variation of the difference in power absorbed as a function of the temperature.

As used herein, the term “apolar wax” means a hydrocarbon-based or silicone apolar wax.

The waxes may be chosen from hydrocarbon-based waxes, fluoro waxes and/or silicone waxes and may be of plant, mineral, animal and/or synthetic origin. For example, the waxes may have a melting point of greater than 45° C.

As wax that may be used in the composition disclosed herein, mention may be made of beeswax, carnauba wax, candelilla wax, paraffin, microcrystalline waxes, ceresin, ozokerite; synthetic waxes, for instance polyethylene waxes and Fischer-Tropsch waxes, and silicone waxes, for instance alkyl or alkoxy dimethicones containing from 16 to 45 carbon atoms.

The composition disclosed herein may contain at least one apolar wax such as a hydrocarbon-based or silicone apolar wax.

The composition may contain at least one apolar hydrocarbon-based wax. As used herein, the term “apolar hydrocarbon-based wax” means a wax containing at least 95% by weight of chemical compounds free of polar groups. The “polar groups” may be well known to those skilled in the art; they may, for example, be ionic or nonionic polar groups chosen from —COOH; —OH; ethylene oxide; propylene oxide; —PO₄; —NHR; and —NR₁R₂ with R₁ and R₂ optionally forming a ring and representing a linear or branched C₁ to C₂₀ alkyl or alkoxy radical.

According to one embodiment, the hydrocarbon-based wax contains at least 95% by weight of compounds free of hetero atoms. As used herein, the term “hetero atom” means an atom other than carbon or hydrogen.

According to one embodiment, the apolar hydrocarbon-based wax contains at least 95% by weight of chemical compounds comprising carbon and hydrogen. These chemical compounds may be chosen from linear or branched saturated alkanes.

According to one embodiment, the apolar wax is chosen from linear hydrocarbon-based waxes.

The linear hydrocarbon-based waxes include ethylene polymers, ethylene copolymers, linear paraffin waxes, and Fischer-Tropsch waxes.

Non-limiting illustrations of hydrocarbon-based waxes that may be mentioned include Fischer-Tropsch waxes, which are also known as polymethylene waxes. They correspond to the formula CₙH₂ₙ₊₂.

According to one embodiment disclosed herein, the wax may be a polymethylene wax, for example the wax Cirebelle® 505 manufactured by the company Sasol, with a melting point equal to 40° C.

The apolar wax may be a polyoxyalkylenated silicone wax, i.e., a silicone comprising at least one oxyalkylene group of the type (—CₓH₂ₓO)ₐ in which x may range from 2 to 6 and a is greater than or equal to 2.

The oxyalkylenated silicones that may be suitable for the uses disclosed herein may be chosen from the compounds of formulae (I), (II), (III), (IV), and (V): 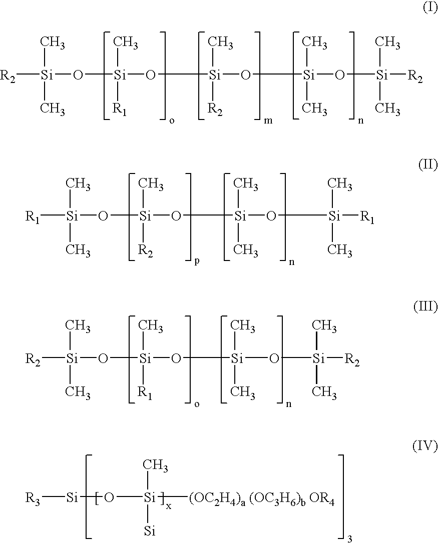
in which formulae (I), (II), (III), and (IV):
R₁, which may be identical or different, is chosen from linear or branched C₁-C₃₀ alkyl radicals and phenyl radicals,
R₂, which may be identical or different, is chosen from C_{c}H_{2c}—O—(C₂H₄O)ₐ(C₃H₆O)_{b}—R₅ radicals and —C_{c}H_{2c}—O—(C₄H₈O)ₐ—R₅ radicals,
R₃ and R₄, which may be identical or different, are chosen from linear or branched C₁ to C₁₂ alkyl radicals, such as methyl radicals,
R₅, which may be identical or different, is chosen from hydrogen, linear or branched alkyl radicals of 1 to 12 carbon atoms, linear or branched alkoxy radicals of 1 to 6 carbon atoms, linear or branched acyl radicals of 2 to 30 carbon atoms, hydroxyl radicals, C₁-C₆ aminoalkoxy radicals optionally substituted on the amine, C₂-C₆ aminoacyl radicals optionally substituted on the amine, aminoalkyl radicals optionally substituted on the amine and on the alkyl chain, C₂-C₃₀ carboxyacyl radicals, groups optionally substituted with one or two substituted aminoalkyl radicals, —NHCO(CH₂)_{d}OH, phosphate groups, and —M, which may be identical or different, is chosen from hydrogen, Na, K, Li, NH₄, and organic amines,
d ranges from 1 to 10,
m ranges from 0 to 20,
n ranges from 0 to 500,
o ranges from 0 to 20,
p ranges from 1 to 50,
a ranges from 0 to 50,
b ranges from 0 to 50, wherein
a plus b is greater than or equal to 2,
c ranges from 0 to 4, and
x ranges from 1 to 100.

Such silicones are described, for example, in U.S. Pat. Nos. 5,070,171; 5,149,765; 5,093,452; and 5,091,493.

The silicones that may be mentioned with respect to at least one embodiment are those of formula (III) in which R₂, which may be identical or different, represents a radical C_{c}H_{2c}—O—(C₂H₄O)ₐ(C₃H₆O)_{b}—R₅, with R₅, a, b, and c being defined as above. In this embodiment, b and c may be equal to 0 and a may range from 1 to 50, such as from 5 to 30 or from 10 to 20.

The low-melting apolar wax may be present in an amount ranging from 1% to 30%, such as from 3% to 20%, by weight relative to the total weight of the composition.

The mass ratio between the polymer particles and the low-melting apolar wax may range from 0.5 to 100, such as from 1 to 50, from 1 to 20, or from 3 to 15.

The composition disclosed herein may also comprise at least one additional wax other than the low-melting apolar wax described above.

The at least one additional wax may have a melting point of greater than or equal to 65° C. It may be chosen from at least one of beeswax, carnauba wax, candelilla wax, paraffin, microcrystalline waxes, ceresin, ozokerite, polyethylene waxes, and Fischer-Tropsch waxes.

According to one embodiment, the apolar wax with a melting point of less than 65° C. (a) and the wax whose melting point is greater than or equal to 65° C. (b) are in a mass proportion (a)/(b) ranging from 30/70 to 55/45, such as from 40/60 to 45/55.

The amount of all the waxes contained in the composition may range from 15% to 35%, such as from 20% to 30%, by weight relative to the total weight of the composition.

The at least one additional wax may be an apolar wax with a melting point of greater than 65° C.; such a wax may be chosen, for instance, from microcrystalline waxes, polyethylene waxes, and paraffin waxes, and mixtures thereof.

The composition disclosed herein may also comprise at least one pasty compound.

Hydrophilic Medium

The composition disclosed herein may comprise a hydrophilic medium comprising water or a mixture of water and of at least one hydrophilic organic solvent, for instance alcohols, for example linear or branched lower monoalcohols containing from 2 to 5 carbon atoms, for instance ethanol, isopropanol, and n-propanol; and polyols, for instance glycerol, diglycerol, propylene glycol, sorbitol, pentylene glycol, and polyethylene glycols; and hydrophilic C₂ ethers and C₂-C₄ aldehydes.

The water or the mixture of water and of at least one hydrophilic organic solvent may be present in the composition disclosed herein in an amount ranging from 0.1% to 99% by weight, such as from 10% to 80% by weight, relative to the total weight of the composition.

Semi-Crystalline Polymer

As used herein, the term “polymers” means compounds containing at least two repeating units, such as at least three repeating units or at least ten repeating units.

As used herein, the term “semi-crystalline polymer” means polymers comprising a crystallizable portion and an amorphous portion in the skeleton and having a first-order reversible change of phase, such as of melting (solid-liquid transition). The crystallizable portion may be either a side chain (or pendent chain) or a block in the skeleton.

When the crystallizable portion of the semi-crystalline polymer is a block of the polymer skeleton, this crystallizable block has a different chemical nature from that of the amorphous blocks. According to the present disclosure, the semi-crystalline polymer is a block copolymer, for example a diblock, triblock, or multiblock copolymer. When the crystallizable portion is a chain that is pendent on the skeleton, the semi-crystalline polymer may be chosen from homopolymers and copolymers.

As used herein, the terms “organic compound” and “having an organic structure” mean compounds containing carbon atoms and hydrogen atoms and optionally hetero atoms such as S, O, N, and P, alone or in combination.

The melting point of the semi-crystalline polymer may be less than 150° C.

The melting point of the semi-crystalline polymer may be greater than or equal to 30° C. and less than 100° C. For example, the melting point of the semi-crystalline polymer may be greater than or equal to 30° C. and less than 60° C.

The at least one semi-crystalline polymer may be solid at room temperature (25° C.) and atmospheric pressure (760 mmHg), with a melting point of greater than or equal to 30° C. The melting point values correspond to the melting point measured using a differential scanning calorimeter (DSC), such as the calorimeter sold under the name DSC 30 by the company Mettler, with a temperature rise of 5 or 10° C. per minute (the melting point under consideration is the point corresponding to the temperature of the most endothermic peak of the thermogram).

The at least one semi-crystalline polymer as disclosed herein may have a melting point that is higher than the temperature of the keratinous support intended to receive the said composition, such as the skin and/or the lips.

The at least one semi-crystalline polymer disclosed herein is capable, alone or as a mixture, of structuring the composition without the addition of a surfactant, filler, or wax.

According to the present disclosure, the at least one semi-crystalline polymer may be soluble in the fatty phase, for example to at least 1% by weight, at a temperature that is higher than its melting point. Besides the crystallizable chains or blocks, the blocks of the polymers are amorphous.

As used herein, the expression “crystallizable chain or block” means a chain or block which, if it were alone, would change from the amorphous state to the crystalline state reversibly, depending on whether one is above or below the melting point. As used herein, a “chain” is a group of atoms, which are pendent or lateral relative to the polymer skeleton. A “block” is a group of atoms belonging to the skeleton, this group comprising one of the repeating units of the polymer.

For example, the polymer skeleton of the at least one semi-crystalline polymer may be soluble in the fatty phase.

In certain embodiments, the crystallizable blocks or chains of the at least one semi-crystalline polymer represent at least 30% of the total weight of each polymer, such as at least 40%. The at least one semi-crystalline polymer containing crystallizable side chains may be chosen from homopolymers and copolymers. The at least one semi-crystalline polymer containing crystallizable blocks may be chosen from block polymers and multiblock polymers. They may be obtained by polymerizing a monomer containing reactive (or ethylenic) double bonds or by polycondensation. When the polymers disclosed herein are polymers containing crystallizable side chains, these side chains may be in random or statistical form.

In certain embodiments, the at least one semi-crystalline polymer is of synthetic origin. According to one embodiment, the at least one semi-crystalline polymer does not comprise a polysaccharide skeleton.

The at least one semi-crystalline polymer that may be mentioned may be chosen from at least one of:
block copolymers of polyolefins of controlled crystallization, whose monomers are described, for example, in European Patent No. EP A 0 951 897,
polycondensates, such as aliphatic polyester polycondensates, aromatic polyester polycondensate, and aliphatic/aromatic polyester polycondensates,
homopolymers or copolymers bearing at least one crystallizable side chain and homopolymers or copolymers bearing in the skeleton at least one crystallizable block, for instance those described in U.S. Pat. No. 5,156,911,
homopolymers or copolymers bearing at least one crystallizable side chain, for example bearing at least one fluoro group, such as those described in the patent document WO A 01/19333.

In the last two cases, the crystallizable side chains or blocks are hydrophobic.

A) Semi-Crystalline Polymers Containing Crystallizable Side Chains

Mention may be made of the semi-crystalline polymers containing crystallizable side chains defined in U.S. Pat. No. 5,156,911 and WO A 01/19333. They are homopolymers or copolymers comprising from 50% to 100% by weight of units resulting from the polymerization of at least one monomer bearing a crystallizable hydrophobic side chain.

These homopolymers or copolymers may be of any nature, provided that they meet the conditions mentioned hereinbelow with, for example, the characteristic of being soluble or dispersible in the fatty phase, by heating above their melting point. They may result:
from the polymerization, for example the free-radical polymerization, of at least one monomer containing at least one reactive or ethylenic double bond with respect to a polymerization, namely a vinyl, (meth)acrylic, or allylic group,

from the polycondensation of at least one monomer bearing co-reactive groups (for example carboxylic acid, sulfonic acid, alcohol, amine, and isocyanate), such as, for example, polyesters, polyurethanes, polyethers, polyureas, and polyamides.

1) In general, the crystallizable units (chains or blocks) of the at least one semi-crystalline polymer disclosed herein may be derived from at least one monomer containing at least one crystallizable block or chain, used for manufacturing semi-crystalline polymers. These polymers may be chosen from homopolymers and copolymers resulting from the polymerization of at least one monomer containing at least one crystallizable chain that may be represented by formula X: 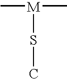
with
M representing an atom of the polymer skeleton,
S representing a spacer, and
C representing a crystallizable group.

The crystallizable chains “—S—C” may be aliphatic or aromatic, and optionally fluorinated or perfluorinated. “S” may represent a group chosen from (CH₂)ₙ, (CH₂CH₂O)ₙ, and (CH₂O), which may be linear or branched or cyclic, with n being an integer ranging from 0 to 22. In certain embodiments, “S” is a linear group. In certain embodiments, “S” and “C” are different.

When the at least one crystallizable chain is a hydrocarbon-based aliphatic chain, it comprises hydrocarbon-based alkyl chains containing at least 11 carbon atoms and not more than 40 carbon atoms, such as not more than 24 carbon atoms. They may be aliphatic chains or alkyl chains containing at least 12 carbon atoms, and they may be C₁₄-C₂₄, such as C₁₆-C₂₂ alkyl chains. When they are fluoroalkyl or perfluoroalkyl chains, they contain at least 11 carbon atoms, at least 6 of which carbon atoms are fluorinated.

As examples of semi-crystalline homopolymers or copolymers containing at least one crystallizable chain, mention may be made of those resulting from the polymerization of at least one of the following monomers: (meth)acrylates of saturated alkyls with the alkyl group being C₁₄-C₂₄; perfluoroalkyl (meth)acrylates with a C₁₁-C₁₅ perfluoroalkyl group; N-alkyl(meth)acrylamides with the alkyl group being C₁₄ to C₂₄ with or without a fluorine atom; vinyl esters containing alkyl or perfluoro(alkyl) chains with the alkyl group being C₁₄ to C₂₄ (with at least 6 fluorine atoms per perfluoroalkyl chain); vinyl ethers containing alkyl or perfluoro(alkyl) chains with the alkyl group being C₁₄ to C₂₄ and at least 6 fluorine atoms per perfluoroalkyl chain; C₁₄ to C₂₄ alpha-olefins such as, for example, octadecene; and para-alkylstyrenes with an alkyl group containing from 12 to 24 carbon atoms.

When the polymers result from a polycondensation, the hydrocarbon-based and/or fluorinated crystallizable chains as defined above are borne by a monomer that may be chosen from diacids, diols, diamines, and diisocyanate.

When the polymers disclosed herein are copolymers, they may contain from 0 to 50% of groups Y or Z resulting from the copolymerization:

a) of Y which is a polar or non-polar monomer or a mixture of the two:

When Y is a polar monomer, it may be chosen from monomers bearing polyoxyalkylenated groups (such as oxyethylenated and/or oxypropylenated groups); hydroxyalkyl (meth)acrylate, for instance hydroxyethyl acrylate; (meth)acrylamide; N-alkyl(meth)acrylamide; N,N-dialkyl(meth)acrylamide such as, for example, N,N-diisopropylacrylamide and N-vinylpyrrolidone (NVP); N-vinylcaprolactam, monomers bearing at least one carboxylic acid group, for instance (meth)acrylic acid, crotonic acid, itaconic acid, maleic acid, and fumaric acid; and from monomers bearing a carboxylic acid anhydride group, for instance maleic anhydride, and mixtures thereof.

When Y is a non-polar monomer, it may be chosen from linear, branched or cyclic alkyl (meth)acrylate esters; vinyl esters; alkyl vinyl ethers; alpha-olefins; styrenes; styrenes substituted with a C₁ to C₁₀ alkyl group, for instance α-methylstyrene; and polyorganosiloxane macromonomers containing vinyl unsaturation.

As used herein, the term “alkyl” means a saturated group, for example of C₈ to C₂₄, except where otherwise mentioned.

b) of Z which is at least one polar monomer. In this case, Z has the same definition as the “polar Y” defined above.

In certain embodiments, the semi-crystalline polymers containing a crystallizable side chain may be chosen from alkyl (meth)acrylate and alkyl(meth)acrylamide homopolymers with an alkyl group as defined above, for example of C₁₄-C₂₄, copolymers of these monomers with a hydrophilic monomer of different nature from (meth)acrylic acid, for instance N-vinylpyrrolidone, and hydroxyethyl (meth)acrylate, and mixtures thereof.

In certain embodiments, the at least one semi-crystalline polymer containing at least one crystallizable side chain has a weight-average molar mass Mp ranging from 5,000 to 1,000,000, such as from 10,000 to 800,000, from 15,000 to 500,000, or from 100,000 to 200,000.

As an example of at least one semi-crystalline polymer that may be used in the composition disclosed herein, mention may be made of the Intelimer® products from the company Landec described in the brochure “Intelimer® polymers”, Landec IP22 (Rev. 4-97). These polymers are in solid form at room temperature (25° C.). They bear crystallizable side chains and have the above formula X.

For example, the Intelimer® product IPA 13-1 from the company Landec may be chosen, which is a polystearyl acrylate with a molecular weight of about 145,000 and a melting point of 49° C.

The at least one semi-crystalline polymer may be those described in Examples 3, 4, 5, 7, and 9 of U.S. Pat. No. 5,156,911, containing a —COOH group, resulting from the copolymerization of acrylic acid and of a C₅ to C₁₆ alkyl (meth)acrylate with a melting point ranging from 20° C. to 35° C., and for example from the copolymerization:

of acrylic acid, of hexadecyl acrylate, and of isodecyl acrylate in a 1/16/3 ratio;

of acrylic acid and of pentadecyl acrylate in a 1/19 ratio;

of acrylic acid, of hexadecyl acrylate, and of ethyl acrylate in a 2.5/76.5/20 ratio;

of acrylic acid, of hexadecyl acrylate, and of methyl acrylate in a 5/85/10 ratio; and

of acrylic acid and of polyoctadecyl methacrylate in a 2.5/97.5 ratio.

It is also possible to use the polymer Structure “O” from National Starch, such as the product described in U.S. Pat. No. 5,736,125, with a melting point of 44° C.

The at least one semi-crystalline polymer may be a semi-crystalline polymer with crystallizable pendent chains comprising fluoro groups, as described in Examples 1, 4, 6, 7, and 8 of patent document WO A 01/19333.

It is also possible to use the semi-crystalline polymers obtained by copolymerization of stearyl acrylate and of acrylic acid or of NVP, as described in U.S. Pat. No. 5,519,063 and European Patent No. EP A 550 745.

It is also possible to use the semi-crystalline polymers obtained by copolymerization of behenyl acrylate and of acrylic acid or of NVP, as described in U.S. Pat. No. 5,519,063 and European Patent No. EP A 055 0745,

B) Polymers Bearing in the Skeleton at Least One Crystallizable Block

Disclosed herein are polymers that are soluble or dispersible in the fatty phase by heating above their melting point. These polymers may be block copolymers comprising at least two blocks of different chemical nature, one of which is crystallizable.

The polymer bearing in the skeleton at least one crystallizable block may be chosen from block copolymers of olefin or of cycloolefin containing a crystallizable chain, for instance those derived from the block polymerization of:

cyclobutene, cyclohexene, cyclooctene, norbornene (i.e., bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,

with ethylene, propylene, 1-butene, 3-methyl-1-butene, 1-hexene, 4-methyl-1-pentene, 1-octene, 1-decene, 1-eicosene, or mixtures thereof,

and for example copoly(ethylene/norbornene) blocks and (ethylene/propylene/ethylidene-norbornene) block terpolymers. Those resulting from the block copolymerization of at least two C₂-C₁₆, such as C₂-C₁₂ α-olefins such as those mentioned above and for example block bipolymers of ethylene and of 1-octene may also be used.

The polymer bearing in the skeleton at least one crystallizable block may be chosen from copolymers containing at least one crystallizable block, the rest of the copolymer being amorphous (at room temperature). These copolymers may also contain two crystallizable blocks of different chemical nature.

Copolymers that may be mentioned are those that simultaneously contain at room temperature a crystallizable block and an amorphous block that are both hydrophobic and lipophilic, sequentially distributed. Mention may be made, for example, of polymers containing one of the crystallizable blocks and one of the amorphous blocks below:

Blocks that are crystallizable by nature, of polyester type, for instance poly(alkylene terephthalate), and polyolefin blocks, for instance polyethylenes and polypropylenes.

Amorphous and lipophilic blocks, for instance: amorphous polyolefins and copoly(olefin)s such as poly(isobutylene), hydrogenated polybutadiene, and hydrogenated poly(isoprene).

As examples of such copolymers containing a crystallizable block and an amorphous block, mention may be made of:
a) poly(ε-caprolactone)-b-poly(butadiene) block copolymers, optionally used hydrogenated, such as those described in the article “Melting behavior of poly(-caprolactone)-block-polybutadiene copolymers” from S. Nojima, Macromolecules, 32, 3727-3734 (1999),
b) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),
c) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997), and
d) the poly(ethylene)-b-poly(ethylethylene) block copolymers cited in the general article “Crystallization in block copolymers” by I. W. Hamley, Advances in Polymer Science, Vol. 148, 113-137 (1999).
   C) Aliphatic Polyester, Aromatic Polyester, and Aliphatic/Aromatic Polyester Polycondensates

The polyester polycondensates may be chosen from aliphatic polyesters. Their molar mass may be greater than or equal to 200 and less than or equal to 10,000, such as greater than or equal to 300 and less than or equal to 5,000, or greater than or equal to 500 and less than or equal to 2,000 g/mol.

The polyester polycondensates may be chosen from polycaprolactones. For example, the polycaprolactones may be chosen from ε-caprolactone homopolymers. The homopolymerization may be initiated with a diol, such as a diol containing from 2 to 10 atoms, such as diethylene glycol, 1,4-butanediol, and neopentyl glycol.

Polycaprolactones may be used for example, those sold under the name CAPA® 240 (melting point of 68° C. and molecular weight of 4,000), CAPA®223 (melting point of 48° C. and molecular weight of 2,000), CAPA® 222 (melting point of 48° C. and molecular weight of 2,000), CAPA® 217 (melting point of 44° C. and molecular weight of 1,250), CAPA® 2125 (melting point of 45° C. and molecular weight of 1,250), CAPA® 212 (melting point of 45° C. and molecular weight of 1,000), CAPA® 210 (melting point of 38° C. and molecular weight of 1,000), and CAPA® 205 (melting point of 39° C. and molecular weight of 830) by the company Solvay, and PCL-300 and PCL-700 by the company Union Carbide.

The CAPA® 2125 whose melting point ranges from 35 to 45° C. and whose molar mass is equal to 1,250 may be mentioned.

The at least one semi-crystalline polymer in the composition disclosed herein may or may not be partially crosslinked, provided that the degree of crosslinking does not interfere with their dissolution or dispersion in the fatty phase by heating above their melting point. It may then be a chemical crosslinking, by reaction with a multifunctional monomer during the polymerization. It may also be a physical crosslinking which may, in this case, be due either to the establishment of bonds of hydrogen or dipolar type between groups borne by the polymer, such as, for example, the dipolar interactions between carboxylate ionomers, these interactions being of small amount and borne by the polymer skeleton; or to a phase separation between the crystallizable blocks and the amorphous blocks borne by the polymer.

In certain embodiments, the at least one semi-crystalline polymer in the composition disclosed herein is non-crosslinked.

In practice, the total amount of the at least one semi-crystalline polymer may be present in an amount ranging from 0.1% to 80%, such as from 0.5% to 40% or from 3% to 30%, by weight relative the total weight of the composition. It may, for example, represent from 5% to 25% by weight of the composition.

Particulate Phase

The composition disclosed herein may contain pigments and/or fillers.

The pigments may be white or colored, mineral and/or organic, and of interference or non-interference type. Among the mineral pigments which may be mentioned are titanium dioxide, optionally surface-treated; zirconium oxide; cerium oxide; zinc oxide; iron oxide (black, yellow, and red); chromium oxide; manganese violet; ultramarine blue; chromium hydrate; and ferric blue. Among the organic pigments that may be mentioned are carbon black, pigments of the type such as organic lakes of barium, strontium, calcium, and aluminium, including those submitted for certification by the Food and Drug Administration (FDA) (e.g., D&C and FD&C) and those exempt from FDA certification, for instance lakes based on cochineal carmine. The pigments may be present in an amount ranging from from 0.1% to 50%, such as from 0.5% to 35% or from 2% to 25%, by weight of active material relative to the total weight of the composition.

The nacreous pigments may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the type mentioned above, as well as nacreous pigments based on bismuth oxychloride. They may be present in an amount ranging from 0 to 25% (of active material), such as from 0.1% to 15% by weight relative to the total weight of the composition (if present). Pigments with goniochromatic properties and/or pigments with a metallic effect as described French Patent Application No. FR 2 842 417, the content of which is incorporated by reference herein, may thus be used.

The fillers may be mineral or organic, and lamellar or spherical. Mention may be made of talc, mica, silica, kaolin, Nylon® powder (Orgasol® from Atochem), poly-β-alanine powder, and polyethylene powder, powders of tetrafluoroethylene polymers (Teflon®), lauroyllysine, starch, boron nitride, hollow microspheres such as Expancel® (Nobel Industrie), Polytrap® (Dow Corning), silicone resin microbeads (for example Tospearls® from Toshiba), precipitated calcium carbonate, magnesium carbonate, magnesium hydrocarbonate, hydroxyapatite, hollow silica microspheres (Silica Beads® from Maprecos), glass microcapsules, ceramic microcapsules, metal soaps derived from carboxylic organic acids containing from 8 to 22 carbon atoms, such as from 12 to 18 carbon atoms, for example zinc stearate, magnesium stearate, and lithium stearate, zinc laurate, and magnesium myristate.

The composition disclosed herein may contain particles that are solid at room temperature, dispersed in the physiologically acceptable medium, introduced into the composition in the form of a colloidal dispersion, as described in Patent Application No. WO 02/39961, the content of which is incorporated by reference herein.

The composition may contain at least one dispersant. The dispersant may serve to protect the dispersed filler or pigment particles against their agglomeration or flocculation. The concentration of dispersant that may be used to stabilize a colloidal dispersion may range from 0.3 to 5 mg/m², such as from 0.5 to 4 mg/m² of surface area of pigment and/or filler particles. This dispersant may be chosen from at least one of surfactants, oligomers, and polymers, comprising at least one functionality having strong affinity for the surface of the particles to be dispersed. For example, they can physically or chemically attach to the surface of the pigments. These dispersants also have at least one functional group that is compatible with or soluble in the continuous medium. For example, at least one of esters of 12-hydroxystearic acid, such as of a C₈ to C₂₀ fatty acid and of a polyol, for instance glycerol or diglycerol are used, such as poly(12-hydroxystearic acid) stearate with a molecular weight of about 750 g/mol, such as the product sold under the name Solsperse® 21 000 by the company Avecia, polyglyceryl-2 dipolyhydroxystearate (CTFA name) sold under the reference Dehymyls PGPH by the company Henkel, and polyhydroxystearic acid such as the product sold under the reference Arlacel® P100 by the company Uniqema.

As other dispersants that may be used in accordance with the present disclosure, mention may be made of quaternary ammonium derivatives of polycondensed fatty acids, for instance Solsperse® 17 000 sold by the company Avecia, and mixtures of polydimethylsiloxane/oxypropylene such as those sold by the company Dow Corning under the references DC2-5185 and DC2-5225 C.

The polydihydroxystearic acid and the 12-hydroxystearic acid esters may be intended for a hydrocarbon-based or fluorinated medium, whereas the mixtures of oxyethylene/oxypropylene dimethylsiloxane may be intended for a silicone medium.

The colloidal dispersion is a suspension of particles which may be of micron size (<10 μm) in a continuous medium. The volume fraction of particles in a concentrated dispersion is from 20% to 40% or greater than 30%, which corresponds to a weight content that may be up to 70% according to the density of the particles.

Additives and Galenical Forms

The composition disclosed herein may also contain at least one cosmetic and/or dermatological active agents such as those conventionally used.

As cosmetic, dermatological, hygiene and/or pharmaceutical active agents that may be used in accordance with certain embodiments, mention may be made of moisturizers, vitamins, essential fatty acids, sphingolipids, and sunscreens. These active agents are used in a usual amount for a person skilled in the art, for example at concentrations of from 0 to 20%, such as from 0.001% to 15%, by weight relative to the total weight of the composition.

The composition may also comprise any other additive usually used in such compositions, such as water, gelling agents, water-soluble dyes, antioxidants, fragrances, preserving agents, and essential oils.

Needless to say, a person skilled in the art will take care to select this or these optional additional compound(s), and/or the amount thereof, such that the advantageous properties of the composition disclosed herein are not, or are not substantially, adversely affected by the envisaged addition.

In one embodiment, the compositions may be prepared in the usual manner by a person skilled in the art. They may be in the form of a cast product, for example in the form of a stick or tube, or in the form of a dish that may be used by direct contact or with a sponge. For example, they find an application as a cast foundation, a cast makeup rouge, a cast eyeshadow, a lipstick, a base or balm to care for the lips, or a concealer product. They may also be in the form of a soft paste or alternatively a more or less fluid gel or cream, or a liquid, packaged in a tube. They may then constitute foundations, lipsticks, antisun products, and skin-coloring products.

The composition disclosed herein may be anhydrous and, in this case, contains less than 5% of water relative to the total weight of the composition.

These compositions for topical application may constitute a cosmetic, dermatological, hygiene and/or pharmaceutical compositions for protecting, treating and/or caring for the face, the neck, the hands and/or the body (for example care creams, antisun oils, and body gels), makeup compositions (for example makeup gels, creams, and sticks), and compositions for artificially tanning and/or for protecting the skin.

The composition disclosed herein may be in the form of a dermatological and/or care composition for the skin and/or the integuments or in the form of an antisun composition or a body hygiene composition, for example in deodorant form. It may then be in uncolored form. It may then be used as a care base for the skin, the integuments, and/or the lips (lip balms, for protecting the lips against the cold and/or sunlight and/or the wind, or a care cream for the skin, the nails and/or the hair).

Another embodiment disclosed herein is a cosmetic process for caring for and/or making up keratin materials, such as the lips, the integuments and/or the skin, which comprises applying a cosmetic composition as defined above to the lips, the integuments and/or the skin.

The compositions disclosed herein may be obtained by heating the various constituents to the melting point of the highest-melting waxes, followed by casting of the molten mixture in a mold (dish or finger stall). They may also be obtained by extrusion, as described in European Patent Application No. EP A 0 667 146.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

### EXAMPLE 1

### Lipstick

Formula A below illustrates one embodiment.

In tests B and C, the low-melting apolar wax was replaced with:
a high-melting apolar wax (microcrystalline wax, formula B)

a low-melting polar wax (stearyl alcohol, formula C)

| | Formula | Comparative | Comparative |
|---|---|---|---|
| Chemical name | A | formula B | formula C |
| Dispersion of acrylate polymer in | 30 | 30 | 30 |
| hydrogenated polyisobutene, sur- | | | |
| face stabilized with Kraton ® | | | |
| G1701 | | | |
| 2-Decyltetradecanoic acid tri- | 2.02 | 2.02 | 2.02 |
| glyceride | | | |
| Dilinoleyl diol dimer/dilinoleyl | 10 | 10 | 10 |
| dimer copolymers | | | |
| Octyldodecanol | 9 | 9 | 9 |
| BHT | 0.07 | 0.07 | 0.07 |
| Mixture of parabens | 0.4 | 0.4 | 0.4 |
| Polycaprolactone of | 9 | 9 | 9 |
| MW 1,250 g/mol | | | |
| Vinylpyrrolidone/eicosene | 6 | 6 | 6 |
| copolymer | | | |
| Microcrystalline wax | 10 | 20 | 10 |
| Polyethylene wax | 2 | 2 | 2 |
| Polymethylene wax of m.p. 40° C. | 10 | / | / |
| Stearyl alcohol | / | / | 10 |
| Pigments | 6.03 | 6.03 | 6.03 |
| Dimethicone-coated silica | 5 | 5 | 5 |
| Fragrance | 0.48 | 0.48 | 0.48 |
| TOTAL | 100 | 100 | 100 |

Synthesis of the Polymer Dispersion:

A dispersion of non-crosslinked copolymer of methyl acrylate and of acrylic acid in an 85/15 ratio, in heptane, was prepared according to the method of Example 1 of European Patent No. EP A 749 746. When the polymerization was complete, hydrogenated polyisobutene was added and the heptane was distilled off under vacuum.

A dispersion of poly(methyl acrylate/acrylic acid) particles surface-stabilized in the hydrogenated polyisobutene with a polystyrene/copoly(ethylene-propylene) diblock copolymer sold under the name Kraton® G1701, having a solids content of 21% by weight and a mean particle size equal to 150 nm, was thus obtained.

Procedure for Preparing the Lipstick

All the starting materials were weighed out in an oil-circulated jacketed heating pan and were then heated with stirring (turbomixer).

After total melting of the materials and homogenization of the mixture, it was ground 5 times in succession on a three-roll mill. The paste obtained was left to stabilize for 24 hours at 20° C. and then packaged in heating bags.

Cosmetic Evaluation (in vivo):

Formula B & Formula C were tested in half-lip tests, in comparison with the control formula A, on 7 women.

The women graded on a scale from 1 to 10 the criteria to be evaluated. After this test, formula A was judged as being glossier, less tacky, more slippery, less dry, and softer than formula B and formula C.

Formula B was also evaluated in a blind test, in comparison with formula A, on six women, the product being applied by a beautician.

Formula A was judged as being more uniform, more adherent and glossier, and as having a stronger color (criteria evaluated by the beautician).

In the comparative formula D below, the following were simultaneously replaced, relative to formula A:
the dispersion of polymer in hydrogenated polyisobutene with hydrogenated polyisobutene alone, and

the low-melting apolar wax with a low-melting polar wax.

| | | Comparative |
|---|---|---|
| Chemical name | Formula A | Formula D |
| Dispersion of acrylate polymer in | 30 | / |
| hydrogenated polyisobutene, surface- | | |
| stabilized with Kraton ® G1701 | | |
| Hydrogenated polyisobutene | / | 30 |
| 2-Decyltetradecanoic acid triglyceride | 2.02 | 2.02 |
| Dilinoleyl diol dimer/dilinoleic dimer | 10 | 10 |
| copolymers (Lusplan DD-DA 5) | | |
| Octyldodecanol | 9 | 9 |
| BHT | 0.07 | 0.07 |
| Mixture of parabens | 0.4 | 0.4 |
| Polycaprolactone of MW 1250 g/mol | 9 | 9 |
| Vinylpyrrolidone/eicosene copolymer | 6 | 6 |
| Microcrystalline wax | 10 | 10 |
| Polyethylene wax | 2 | 2 |
| Polymethylene wax of m.p. 40° C. | 10 | / |
| Hydrogenated cocoglycerides | / | 10 |
| Pigments | 6.03 | 6.03 |
| Dimethicone-coated silica | 5 | 5 |
| Fragrance | 0.48 | 0.48 |
| TOTAL | 100 | 100 |

The procedure was the same as that described above.

Cosmetic Evaluation (in vivo):

According to the same protocol as that described above, these two formulae were prepared in half-lip tests. The application of formula D was judged as being greasier, the deposit was finer, and the migration over time was markedly more pronounced.

Evaluation (in vitro):

The two formulae A and D were tested in vitro according to the wear test described above.

The results were as follows:

| | Formula A | Formula D |
|---|---|---|
| | (dispersion + apolar wax | (hydrogenated polyisobutene + |
| | of low m.p.) | polar wax of low m.p.) |
| Resistance to | 91.48 | 63.19 |
| pressure | | |
| Resistance to | 53.96 | 29.14 |
| pressure + | | |
| wiping | | |

1. A cosmetic composition comprising
polymer particles dispersed in a fatty phase, and
at least 5% by weight, relative to the total weight of the composition, of at least one apolar wax with a melting point of less than 65° C.

2. The composition according to claim 1, wherein the fatty phase comprises at least 5% by weight of at least one non-volatile hydrocarbon-based oil, relative to the total weight of the composition.

3. The composition according to claim 1, wherein the fatty phase is free of volatile oil or comprises less than 50% by weight of at least one volatile oil, relative to the weight of the fatty phase.

4. The composition according to claim 1, wherein:
the dynamic viscosity of the composition measured at 25° C., using a Mettler RM 180 rotary viscometer, ranges from 0.1 to 120 Pa.s, and
the amount of wax is greater than or equal to 15% by weight relative to the total weight of the composition.

5. The composition according to claim 1, wherein the at least one apolar wax has a melting point ranging from 35° C. to 55° C.

6. The composition according to claim 1, wherein the at least one apolar wax has a melting point ranging from 35° C. to 50° C.

7. The composition according to claim 6, wherein the at least one apolar wax has a melting point ranging from 35° C. to 45° C.

8. The composition according to claim 1, wherein the polymer is such that, when it is dispersed in the composition in sufficient amount, the composition is capable of forming a deposit that has a wear index of greater than or equal to 30%.

9. The composition according to claim 1, wherein the polymer is such that, when it is dispersed in the fatty phase in sufficient amount, the mean gloss at 20° of a deposit of the composition, once spread onto a support, is greater than or equal to 30 out of 100.

10. The composition according to claim 1, wherein the at least one apolar wax is a linear hydrocarbon-based wax.

11. The composition according to claim 1, wherein the at least one apolar wax is a hydrocarbon-based wax comprising linear alkanes.

12. The composition according to claim 11, wherein the at least one apolar wax is a Fischer-Tropsch wax.

13. The composition according to claim 1, further comprising at least one wax having a melting point greater than or equal to 65° C.

14. The composition according to claim 13, wherein the at least one wax having a melting point greater than or equal to 65° C. is chosen from beeswax, carnauba wax, candelilla wax, paraffin, microcrystalline waxes, ceresin, ozokerite, polyethylene waxes, and Fischer-Tropsch waxes.

15. The composition according to claim 13, wherein the at least one wax having a melting point greater than or equal to 65° C. is apolar.

16. The composition according to claim 13, wherein the at least one apolar wax having melting point less than 65° C. (a) and the at least one wax having melting point greater than or equal to 65° C. (b) are in a mass proportion (a)/(b) ranging from 30/70 to 55/45.

17. The composition according to claim 16, wherein the mass proportion (a)/(b) ranges from 40/60 to 45/55.

18. The composition according to claim 13, wherein the at least one apolar wax with a melting point amount of less than 65° C. and the at least one wax having a melting point greater than or equal to than 65° C. are present in the composition in a combined amount ranging from 15% to 35% by weight, relative to the total weight of the composition.

19. The composition according to claim 18, wherein the waxes are present in an amount ranging from 20% to 30% by weight relative to the total weight of the composition.

20. The composition according to claim 1, wherein the viscosity of the composition ranges from 5 to 20 Pa.s.

21. The composition according to claim 20, wherein the polymer particles are solid and insoluble in the liquid fatty phase at a temperature of 25° C.

22. The composition according to claim 1, wherein the polymer is not a wax.

23. The composition according to claim 1, wherein the polymer particles have a mean size ranging from 5 to 800 nm.

24. The composition according to claim 1, wherein the polymer is film-forming.

25. The composition according to claim 24, wherein the polymer is a hydrocarbon-based polymer.

26. The composition according to claim 1, wherein the composition comprises less than 10% of least one silicone oil, by weight relative to the total weight of the composition.

27. The composition according to claim 26, wherein the composition comprises less than 1% of least one silicone oil, by weight relative to the total weight of the composition.

28. The composition according to claim 1, wherein the composition comprises less than 10% of at least one volatile oil, by weight relative to the total weight of the composition.

29. The composition according to claim 28, wherein the composition comprises less than 1% of at least one volatile oil, by weight relative to the total weight of the composition.

30. The composition according to claim 1, wherein the polymer particles are insoluble in water-soluble alcohols.

31. The composition according to claim 1, wherein the polymer particles are chosen from polyurethanes, polyurethane-acrylics, polyureas, polyureas/polyurethanes, polyester-polyurethanes, polyether-polyurethanes, polyesters, polyester amides, fatty-chain polyesters, alkyds, acrylic polymers, acrylic copolymers, vinyl polymers, vinyl copolymers, acrylic-silicone copolymers, polyacrylamides, silicone polymers, and fluoro polymers, and mixtures thereof.

32. The composition according to claim 1, wherein the polymer is chosen from meth(acrylic)/methacrylate copolymers.

33. The composition according to claim 32, wherein the polymer is chosen from acrylic/acrylate copolymers such that the mass ratio of the acrylic units and of the acrylate units ranges from 0.1% to 40%.

34. The composition according to claim 33, wherein the polymer is chosen from acrylic/acrylate copolymers such that the mass ratio of the acrylic units and of the acrylate units ranges from 5% to 20%.

35. The composition according to claim 1, wherein the polymer is present, as solids, in an amount ranging from 5% to 40% by weight relative to the total weight of the composition.

36. The composition according to claim 35, wherein the polymer is present, as solids, in an amount ranging from 8% to 30% by weight relative to the total weight of the composition.

37. The composition according to claim 1, further comprising at least one stabilizer chosen from block polymers, grafted polymers, and random polymers.

38. The composition according to claim 37, wherein the at least one stabilizer is chosen from grafted-block and block polymers comprising at least one block resulting from the polymerization of diene and at least one block of a vinyl polymer.

39. The composition according to claim 38, wherein the at least one stabilizer is a diblock polymer.

40. The composition according to claim 1, wherein the composition comprises at least 5% by weight of at least one apolar or sparingly polar oil, relative to the total weight of the composition.

41. The composition according to claim 40, wherein the at least one apolar or sparingly polar oil is a hydrocarbon-based oil.

42. The composition according to claim 41, wherein the at least one apolar or sparingly polar hydrocarbon-based oil is present in an amount ranging from 5% to 80% by weight relative to the total weight of the composition.

43. The composition according to claim 40, wherein the at least one apolar or sparingly polar hydrocarbon-based oil is present in an amount ranging from 10% to 60% by weight relative to the total weight of the composition.

44. The composition according to claim 43, wherein the at least one apolar or sparingly polar hydrocarbon-based oil is present in an amount ranging from 15% to 30% by weight relative to the total weight of the composition.

45. The composition according to claim 41, wherein the at least one apolar or sparingly polar hydrocarbon-based oil is an apolar hydrocarbon-based oil.

46. The composition according to claim 45, wherein the at least one apolar hydrocarbon-based oil is chosen from oils with a molar mass ranging from 300 to 900 g/mol.

47. The composition according to claim 46, wherein the at least one apolar hydrocarbon-based oil is chosen from oils with a molar mass ranging from 350 to 800 g/mol.

48. The composition according to claim 46, wherein the at least one apolar hydrocarbon-based oil is chosen from at least one linear or branched hydrocarbon.

49. The composition according to claim 48, wherein the at least one linear or branched hydrocarbon is chosen from liquid paraffin, liquid petroleum jelly, liquid naphthalene, hydrogenated polyisobutene, isoeicosane, squalane, and decene/butene copolymers.

50. The composition according to claim 1, wherein the composition comprises from 2% to 30%, by weight relative to the total weight of the composition, of at least one oil with a molar mass ranging from 650 to 10,000 g/mol.

51. The composition according to claim 50, wherein the composition comprises from 5% to 25%, by weight relative to the total weight of the composition, of the at least one oil with a molar mass ranging from 650 to 10,000 g/mol.

52. The composition according to claim 50, wherein the molar mass of the at least one oil ranges from 750 to 7,500 g/mol.

53. The composition according to claim 1, further comprising at least one pulverulent dyestuff chosen from pigments, nacres, and flakes.

54. The composition according to claim 53, further comprising at least one dispersant chosen from poly(12-hydroxystearic acid stearate), poly(12-hydroxystearic acid), and diglyceryl 2-dipolyhydroxystearate.

55. The composition according to claim 1, wherein the composition is in anhydrous form.

56. The composition according to claim 1, wherein the composition is in the form of a care and/or makeup product for the skin and/or the lips.

57. The composition according to claim 1, wherein the composition is in the form of at least one product chosen from foundations, makeup rouges, eyeshadows, lipsticks, lipcare bases, lipcare balms, concealer products, eyeliners, and mascaras.

58. A method of obtaining a glossy, migration-resistant makeup with good staying power comprising
applying to the skin, lips, and/or integuments a cosmetic composition comprising
polymer particles dispersed in a fatty phase, and
at least 5% by weight, relative to the total weight of the composition, of at least one apolar wax with a melting point of less than 65° C.,
wherein said polymer particles and said at least one apolar wax are present in the composition in an amount sufficient to obtain a glossy, migration-resistant makeup with good staying power.
